(19) United States
(12) Patent Application Publication
(10) Pub. No.: US 2006/0282028 A1
(43) Pub. Date: Dec. 14, 2006

(54) Wound Management System

(76) Inventors: Pamela A. Howard, Allentown, PA (US); Eben Howard, Allentown, PA (US)

Correspondence Address: CAHN & SAMUELS LLP, 2000 P STREET NW, SUITE 200, WASHINGTON, DC, 20036, US

(21) Appl. No.: 11/423,514

(22) Filed: Jun. 12, 2006

Related U.S. Application Data
(60) Provisional application No. 60/689,082, filed on Jun. 10, 2005.

(51) Int. Cl. A61F 5/00

(52) U.S. Cl. 602/2

(57) ABSTRACT

The invention includes a wound dressing having a plurality of layers, at least one conduit system, and means for assisting in the healing process. Exemplary means for assisting in the healing process includes a light source that in at least one embodiment provides ultraviolet light and a vibrational energy source such as a transducer. In at least one exemplary embodiment the wound dressing includes measurement means for measuring temperature or pH, and in at least one further exemplary embodiment providing signals to a controller for adjusting the measured parameter in the wound dressing and/or wound site.

This application claims the benefit of U.S. provisional application Ser. No. 60/689,082, filed on Jun. 10, 2005, which is incorporated herein by reference.

### I TECHNICAL FIELD

The present invention is directed to a wound management system having an integral wetting system for maintaining the wound in a moist condition and the capability to deliver at least one additional medical beneficial treatment.

### II BACKGROUND OF THE INVENTION

It has been found that regulation of the moisture in wound beds helps wounds heal faster. Numerous types of wound dressings have been developed that are designed to maintain a favorable moist environment. For example, an occlusive dressing is designed to keep a wound from being exposed to air, thus reducing evaporation. Many dressings, such as those disclosed in U.S. Pat. No. 6,153,214 (Horsler) and U.S. Pat. No. 5,695,777 (Donovan et al.) incorporate a water-swellable fiber or intermediate hydrogel layer to promote localized areas containing moisture.

However, these dressings are limited in the kind of fluid used to fill the fiber or layer, and, if fluid must be added to the dressing, then the dressings must be constantly tended. Further, pouring water, antibiotic solution, or some other solution on the dressing results in variable wetting and dry spots, both of which may adversely affect wound healing and increase the risk of wound infection and desiccation.

To address uniform wetting, U.S. Pat. No. 6,162,960 (Klein) discloses a compression sponge for dressing a wound having at least one uniform bibulous layer to wick or absorb and retain liquid. The uniform liquid distribution throughout the entire layer provides uniform pressure distribution of any externally applied pressure on the dressing. This type of dressing also requires constant tending, in practice, because it develops dry spots. This type of dressing also is inefficient and ineffective for applying many solutions containing certain therapeutic substances.

A dressing similar to that described in the Klein patent is the Acticoat® Burn Dressing (Westaim Biomedical, Exeter, N.H., U.S.A.). The Acticoat® Burn Dressing includes silver-coated outer layers to protect against infection while the inner core maintains moisture in the dressing. This dressing requires that sterile water be poured on it regularly to provide for continued moisture to be present within the dressing's inner core material. Usually, the Acticoat® Burn Dressing requires water to be added every four to six hours.

The inventors have found that failing to keep the entire wound area moist or wet leads to increased risk of infection and/or desiccation of the wound area. To date, no satisfactory dressing has been developed for difficult wounds, such as deep burns or chronic wounds including decubitus ulcers, venous stasis sores, radiation ulcers, pressure sores, ischemic ulcers or diabetic ulcers. Treatment of these wounds remains a very long and expensive process requiring staff to continually and/or frequently monitor the moisture level of a dressing and to add fluid as needed.

An increase in temperature in a wound site typically is the first sign of an infection beginning in the wound. In addition, the temperature can impact the effectiveness of the medication that is applied to a wound. Another factor that impacts the effectiveness of wound healing is the pH of the wound and dressing, because, for example, different medications have pH ranges in which they are more effective for treatment. Certain types of wounds such as chemical burns need to maintain a pH of the skin about 5.5 to keep the pH closer to normal for the skin may help avoid ongoing injury from the pH of the chemical.

Notwithstanding the usefulness of the above-described methods, a need still exists for a dressing that maintains a moist environment with little monitoring by the medical staff required while improving the healing rate of the wound area.

### SUMMARY OF THE INVENTION

A wound management system in accordance with at least one embodiment the present invention may be used for protection of wounds, for infection control, for control of conditions (such as water content, osmolarity, pH, salt concentration, oxygen concentration) in wounds, for thermal regulation, for delivery of bioactive substances (such as nutrients; antibiotics, vasodilatants, vasoconstrictors, steroids and other bioactive substances; growth factors, hormones, enzymes, stimulants and other biomolecules; sulfamylon; silver) into wounds, for removal of harmful substances from wounds, and for protection and support of the human cells proliferating in wounds.

The primary function of the wound management system according to at least one embodiment of the present invention is to occlude the wound, to control its environment, and to promote healing by maintaining the wound area at a certain pre-determined level of moisture content. The wound management system in at least one embodiment also uniformly distributes moisture or fluid through the wound area, and prevents or reduces the size and number of dry spots covering the wound. In at least one embodiment, the wound management system provides vibrational energy such as ultrasound or kinetics. In at least one embodiment, the wound management system distributes ultraviolet light in the dressing to kill bacteria and/or improve the healing of the wound.

The wound management system according to at least one embodiment of the invention promotes formation of granulation tissue in the wound bed, facilitate migration of epidermal cells, and help to control infections. This leads to faster closure of the wound, less pain, and less scarring.

In at least one embodiment, the invention includes a wound management system including a dressing having a first layer, a second layer abutting the first layer, and a third layer abutting the second layer; an irrigation system in communication with the second layer; and means for treatment in communication with at least one of the second layer and the irrigation system.

In at least one embodiment, the invention includes a wound management system including a dressing having a first layer, a second layer abutting the first layer, and a third layer abutting the second layer; an irrigation system in communication with the second layer; a light distribution system in communication with the dressing, the light distribution system including a light source providing light having a wavelength between 3 nm and 380 nm; and a vibrational energy source in communication with the dressing.

In at least one embodiment, the invention includes a wound management system including treating means for treating the wound, wound covering means for covering the wound site, seepage preventing means for preventing seepage from the wound site to the surrounding environment, feedback means for providing feedback based on conditions within the wound covering means, wetness maintaining means for maintaining wetness within the wound covering means, and moisture supplying means for providing moisture to the wetness maintaining means.

The accompanying drawings show illustrative embodiments of the invention from which these and other of the objectives, novel features and advantages will be readily apparent. Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements. The use of cross-hatching and shading within the drawings is not intended as limiting the type of materials that may be used to manufacture the invention.

FIG. 1A illustrates a schematic cross-section of an exemplary embodiment of the invention.

FIG. 1B depicts a top view of the exemplary embodiment illustrated in FIG. 1A.

FIG. 1C illustrates a side view of the exemplary embodiment depicted in FIG. 1B with an alternative embodiment of the invention.

FIG. 1D illustrates a partial cross-section taken at 1D-1D of the embodiment depicted in FIG. 1B.

FIG. 1E depicts a block diagram of an exemplary embodiment according to the invention.

FIG. 2A illustrates a cross-section view of an exemplary embodiment of the invention illustrating an exemplary irrigation system configuration.

FIG. 2B depicts a side view of the exemplary embodiment illustrated in FIG. 2A.

FIGS. 3A-3C illustrate cross-section views of exemplary embodiments according to the invention.

FIG. 3D depicts a top view of a portion of an exemplary embodiment according to the invention.

FIG. 4A illustrates a cross-section view of an exemplary embodiment according to the invention.

FIG. 4B depicts a top view of a portion of an exemplary embodiment according to the invention.

FIG. 5A illustrates a block diagram of an exemplary embodiment according to the invention.

FIG. 5B illustrates a block diagram of an exemplary embodiment according to the invention.

FIGS. 6A and 6B depict top views of different exemplary conduit configurations for irrigation systems according to the invention.

FIG. 7 illustrates a block diagram of an exemplary embodiment according to the invention.

FIG. 8 depicts a top view of an exemplary embodiment according to the invention.

FIGS. 9A and 9B illustrate side views of different exemplary hole patterns for conduits according to the invention.

FIG. 10 depicts a top view of an exemplary embodiment according to the invention.

### V. DETAILED DESCRIPTION OF THE INVENTION

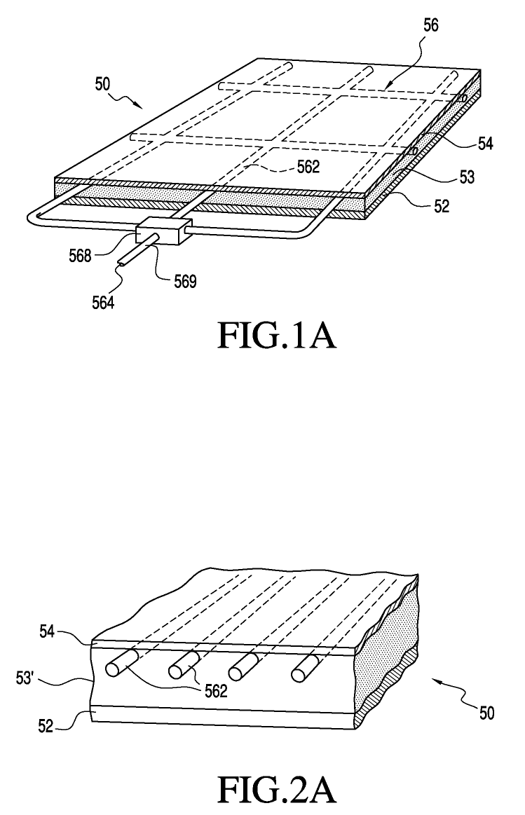

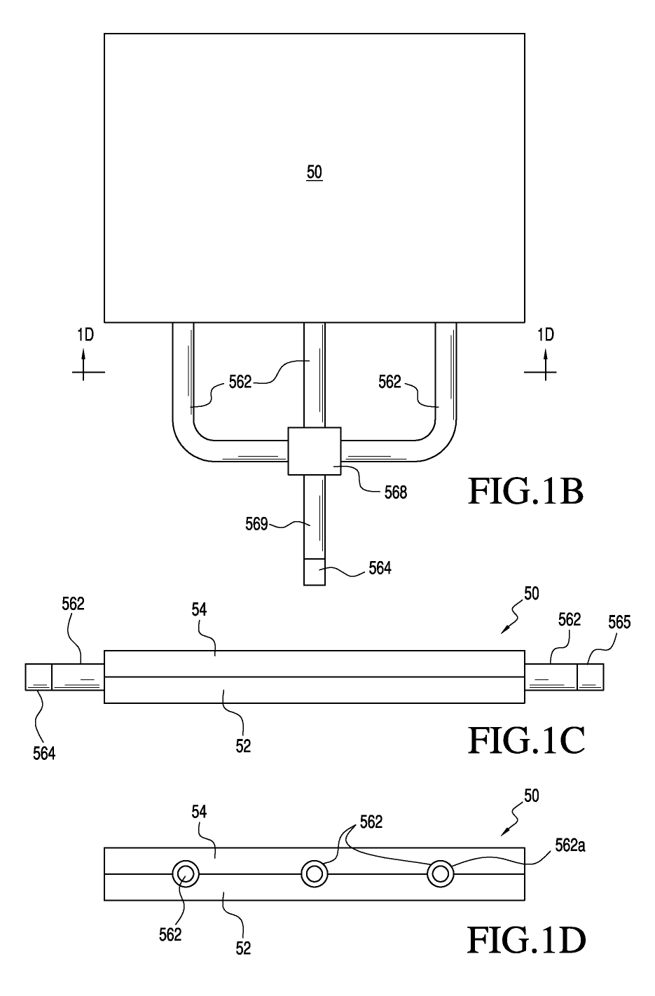

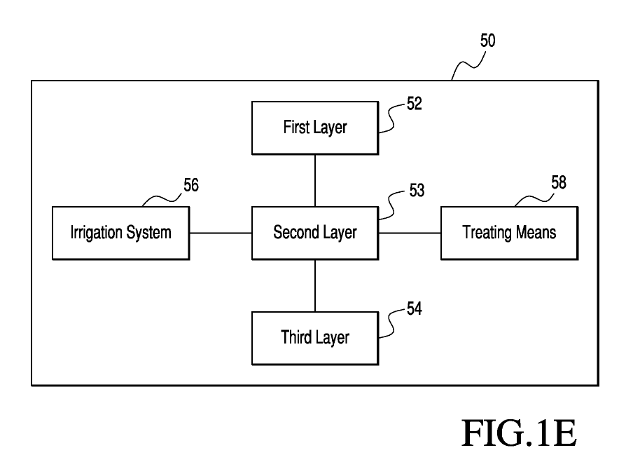

The exemplary embodiment of the present invention as illustrated, for example, in FIGS. 1A-E is a wound management system having a dressing (or bandage) 50 and an irrigation system (or means for maintaining wetness within the dressing) 56 in communication with the dressing 50. The illustrated dressing 50 includes a first layer (or means for covering a wound) 52 for contacting the wound area, a second (or middle) layer 53, and a third or outer layer (or means for preventing seepage from the dressing) 54 that preferably is fluid impermeable. The means for maintaining wetness within the dressing may in at least one embodiment include the second layer 53 in addition to the irrigation system 56. The second layer 53 provides structure and support for the irrigation system 56 to keep it in place within the dressing 50 while decreasing added compression forces on the wound through the first layer 52. This arrangement allows the first layer 52 and the third layer 54 to be thin as is typical in bandages. One of ordinary skill in the art will appreciate based on this specification that an additional layer(s) may be present in the dressing 50.

The wound management system of the present invention is suitable for use with any wound for which it may be physiologically or therapeutically beneficial to maintain moist, or for which it may be desirable to provide a fluid exchange system.

The irrigation system 56 is disposed in the middle layer 53 between the first layer 52 and the third layer 54. In an embodiment where the middle layer 53 is omitted, the irrigation system 56 is disposed between the first layer 52 and the third layer 54, or alternatively the irrigation system 56 may be within the first layer 52. An exemplary irrigation system 56 preferably includes a series of interconnected conduits 562 or the like, each conduit 562 having one or more small pores (or holes) 5622 for providing fluid communication with the first layer 52. The irrigation system 56 is configured to provide one or more fluid flow paths for any therapeutically or physiologically desirable or beneficial fluid, including but not limited to air, oxygen, water, an isotonic solution, a hypertonic solution, or the like.

In exemplary embodiments of the invention, the irrigation system 56 provides generally uniform distribution of fluid throughout the entire first layer 52. In most exemplary embodiments of the invention, the irrigation system 56 provides uniform distribution of fluid across the area of the first layer 52 in contact with or near the wound.

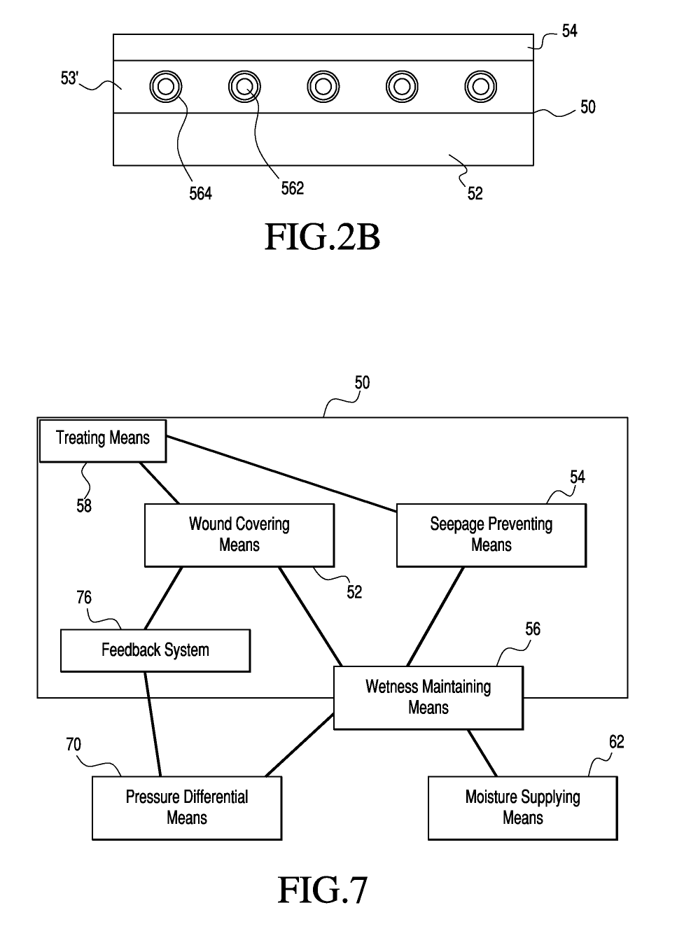

The dressing 50 may also optionally include a moisture retention (or reservoir) layer 53′ as the middle layer positioned between the first layer 52 and the third layer 54 as illustrated in FIGS. 2A and 2B. The moisture retention layer 53′ may also serve to position and/or hold the irrigation system 56 in place within the dressing 50.

The wound management system as illustrated in the exemplary embodiment shown in FIG. 1E includes a means for treating 58 such as a vibrational energy source or system and an ultraviolet light system. The treating means 58 as described can be present within the dressing 50, partially present or external to the dressing 50.

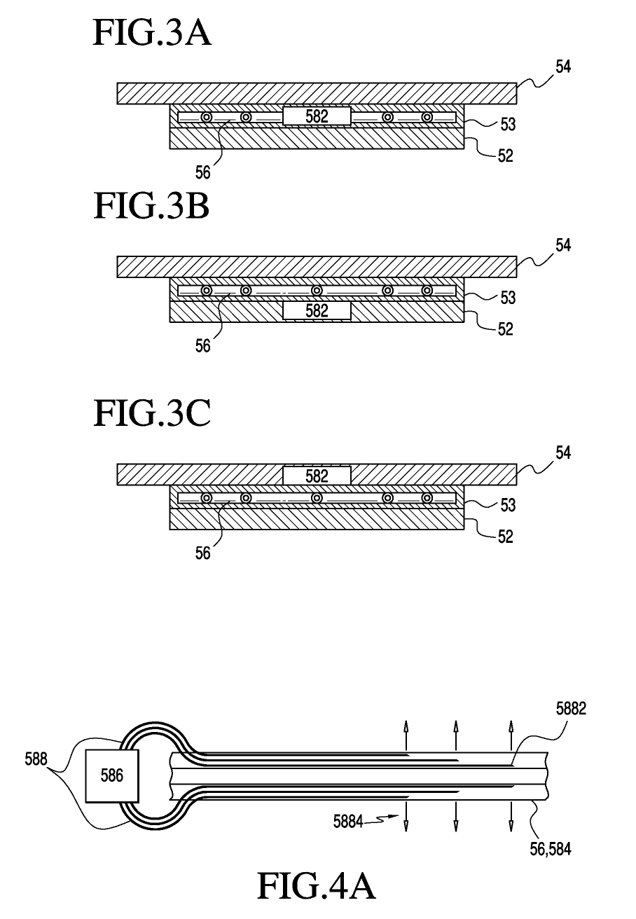

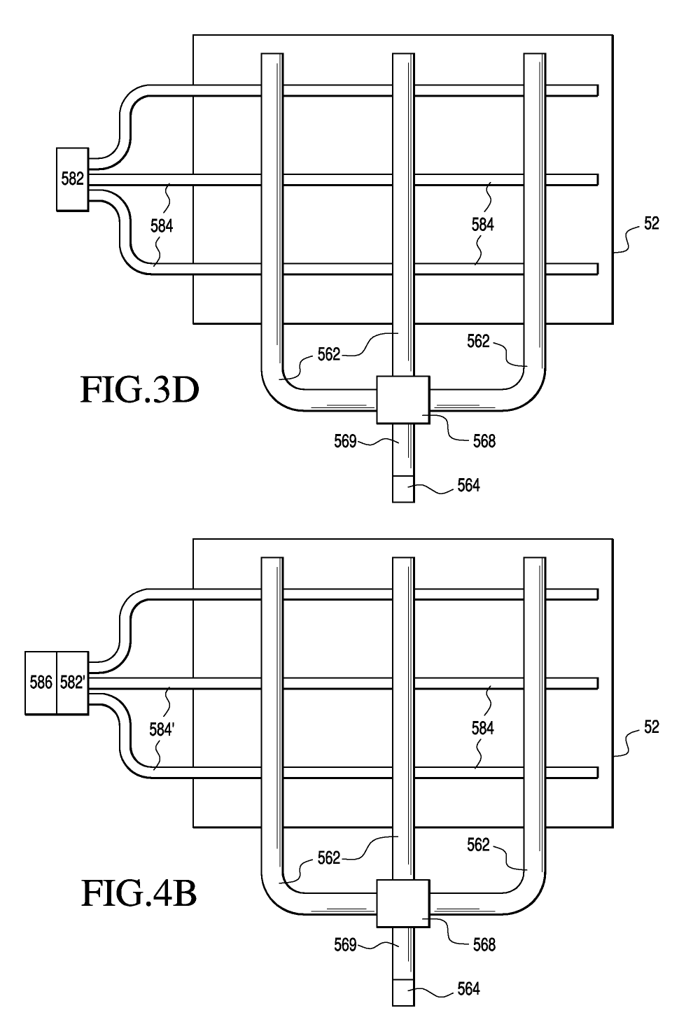

The vibrational energy source preferably includes a transducer 582 capable of creating energy waves to vibrate the wound site to assist with the delivery of medication to the wound site as illustrated, for example, in FIGS. 3A-3D. The energy waves may be provided directly from the transducer 582 where the transducer 582 is present in the second layer 53 (FIG. 3A), in the first layer 52 (FIG. 3B), or in the third layer 54 (FIG. 3C). Alternatively, the energy waves may be supplied to the dressing 50 and the wound through a network of gel or other pliable material filled conduits that form a conduit system 584 similar to the irrigation system 56 examples provided in this specification as illustrated, for example, in FIG. 3D, which illustrates the dressing 50 without the second layer 53 or the third layer 54. The material may alternatively be present in the walls of the conduit. The transducer 582 preferably is matched with the material in the conduit system 584. One of ordinary skill in the art will appreciate based on this disclosure that a variety of orientations and layouts of the conduit system 584 relative to the irrigation system 56 are possible. An advantage to the external transducer 582 is that one transducer may be reusable with multiple dressings. The transducer 582 in at least one embodiment is powered by a power source such as a battery (not illustrated).

The ultraviolet light system in at least one embodiment is incorporated into the conduit of the irrigation system 56 and/or the conduit network 584 of the vibrational energy system as illustrated, for example, in FIG. 4A. The conduit preferably includes fiber optic light distribution system with a plurality of optical fibers 588 running different lengths along the conduit such that an area of interest is likely to be exposed to the light. As illustrated in FIG. 4A, each optical fiber 588 connects to a light source 586 at one end and at the other end is free to provide a light path from the conduit into the wound area and/or dressing 50. The light source 586 may comprise any light source capable of supplying a dosage of light having one or more wavelengths and/or center frequencies to the fiber optic light distribution system such as one or more lasers, light emitting diodes, filtered white light sources, etc. The wavelength of the light preferably is in a range from about 3 nm to about 380 nm including the end points.

As shown in FIG. 4A, each optical fiber 588 is preferably cleaved or otherwise cut so as to have an angled fiber/air interface 5882. Each angled fiber/air interface 5882 is adapted to redirect, for example, via refraction, light traveling within one of the optical fibers 588 radially away from the optical fiber and into the wound. For example, depending on the index of refraction of the optical fiber 588 employed, a fiber/air interface 5882 of about 45 degrees (from the optical or central axis of the fiber) may refract light at an approximately a 90 degree angle (from the optical or central axis of the fiber), as shown in FIG. 4A by light rays 5884. Other fiber/air interface angles and/or angles of refraction may be employed. The free ends of the optical fibers 588 may aim radially out from the conduit in at least one exemplary embodiment. The optical fibers 588 may be “staggered” as shown to allow each fiber to transmit light energy into the wound. Other configurations may be employed. In one embodiment, a 360 degree lateral light distribution is provided. Other types of light distribution may be provided.

Each optical fiber 588 may include any conventional optical fiber, examples include a single node, multi-node, glass, plastic, or fiber. In at least one embodiment of the invention, each optical fiber 588 includes a multi-mode, plastic optical fiber. Plastic optical fibers typically are less likely to break if bent when compared to glass fibers. Bending may occur when the fiber optic light distribution system is bent within a wound area as described in the specification. In at least one exemplary embodiment, the optical fibers 588 form part of at least one of the first layer 52, the second layer 53, and the third layer 54.

An exemplary embodiment in FIG. 4B includes a transducer 582′ that preferably is able to pass light originating from the light source 586 into the gel filled conduit system 584′. In this embodiment, the conduit system 584′ is shielded (or opaque) external to the dressing 50. Inside the dressing 50, light will diffuse from the conduit system 584′ into the dressing layers and/or the wound.

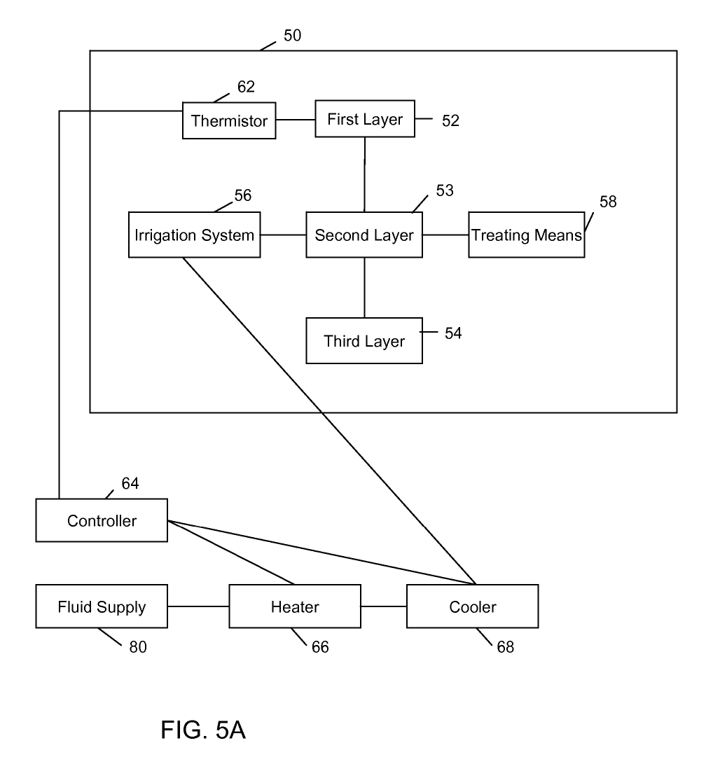

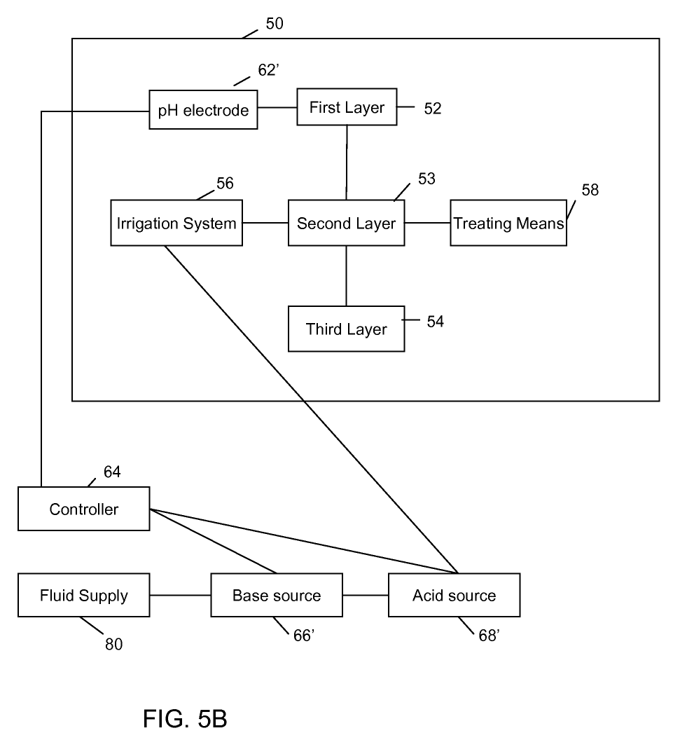

An exemplary embodiment of the wound management system illustrated in FIG. 5A incorporates a thermistor 62 into the dressing 50, although illustrated as being in the first layer 52 the thermistor 62 may be present in the second layer 53 or the third layer 54, to provide a signal indicative of the temperature of the wound area to a controller 64. The controller 64 determines based on the signal whether the temperature of the wound site needs to be adjusted and by how much, or alternatively whether a certain threshold has been exceeded and whether an alarm or other notification should be provided to medical personnel. The controller 64 preferably controls temperature adjustment devices such as a heater 66 to heat fluid being provided to the dressing or a refrigerant system (or cooler) 68 to cool fluid being provided to the dressing until the desired temperature range is reached in the wound dressing. Alternatively, the controller may be programmed to provide a heating and cooling regiment to facilitate medical treatment. The controller in at least one embodiment also controls the light source 586.

An exemplary embodiment of the wound management system illustrated in FIG. 5B incorporates a pH electrode system 62′ into the dressing 50, although illustrated as being in the first layer 52 the pH electrode system 62′ may be present in the second layer 53 or the third layer 54, to provide a signal indicative of the pH of the wound area to a controller 64. The pH electrode system 62′ in at least one embodiment include a plurality of electrodes spread about the wound dressing to obtain an indication of the pH within the wound site. The controller 64 determines based on the signal whether the pH of the wound site needs to be adjusted and by how much, or alternatively whether a certain threshold has been exceeded and whether an alarm or other notification should be provided to medical personnel. The controller 64 in the embodiment illustrated in FIG. 5B controls pH adjustment devices such as an acid source 66′ to lower the pH level in the wound site by increasing the acidity of the fluid being provided to the dressing or a base source 68′ to raise the pH level in the wound site by providing a solution to fluid being sent to the dressing that has a higher pH until the desired pH range is reached in the wound dressing. Alternatively, the controller may be programmed to provide a regiment having different pH levels at different points to facilitate medical treatment. Based on this description, the temperature and pH control embodiments may be incorporated together into one wound dressing. An alternative way to test the pH is to have a vacuum withdraw fluid from the wound site at predetermined times and to test the fluid external to the wound site, with the resulting readings being provided to a controller 64 and/or medical personnel.

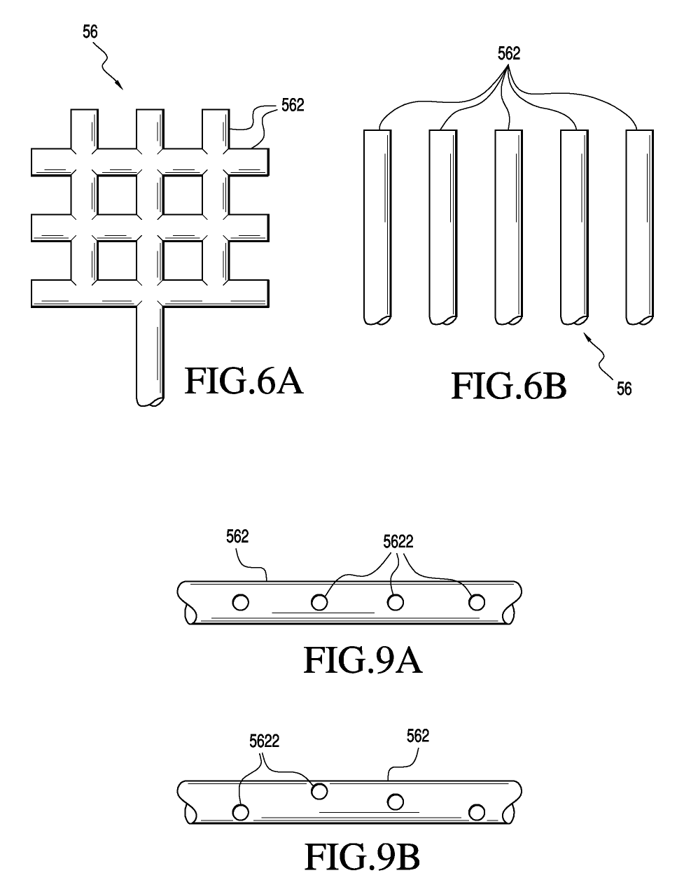

In accordance with the present invention, the irrigation system 56 may be variously configured. In the configuration shown in FIG. 6A, the conduits 562 communicate with one another within the bandage 50 to form a network of conduits. In exemplary embodiments of the invention, the portion of the conduit that is within the dressing 50 includes one or more holes 5622 or the like so that fluid may be transferred between the conduits 562 and the first layer 52. More preferably, the holes 5622 are located from 90 degrees to 270 degrees on conduits 562 where zero degrees is the top 562a and the portion of each conduit 562 radially farthest from the first layer 52 as illustrated in FIG. 1D. Even more preferably, the holes 5622 are located from 110 degrees to 250 degrees on conduit 562. The irrigation system 56 also preferably includes one or more access ports 564 for delivering fluid to the conduit 562 as illustrated, for example, in FIGS. 1B and 3D. As described in more detail below, the preferred access port 564 includes a Luer-type fitting (not shown) for connecting to another conduit, to a syringe, or the like. As shown in FIG. 1B, the irrigation system 56 may also include a manifold (or hub) 568 or the like so that fluid may be administered through a single conduit 569 and distributed throughout the irrigation system 56.

In the configuration illustrated in FIGS. 2A and 6B, the irrigation system 56 includes a series of separate conduits 562. One or both ends of the conduit 562 may include an access port 564. FIG. 2A also shows the alternative reservoir layer 53′ for retaining moisture within the dressing system 50.

The dimensions for this wound dressing can be varied to suit any particular situation or purpose, and can fluctuate greatly depending on the size and number of layers used to form the dressing.

Fluid may be supplied to the irrigation system 56 using any of a number of devices 80 and methods known to those skilled in the art. The fluid supply 80 may be, for example, a syringe or an IV pump connected to the system and used to inject fluid into the system. Liquid is supplied to the irrigation system 56 by establishing a pressure differential between the source of the fluid and the other end of the system. A pressure differential may be established using gravity, pressure or a pump, a vacuum, or any other mechanism for moving fluids from one place to another. Any of the ways for establishing the pressure differential may be means for providing pressure differential 70 as depicted in FIG. 1E. Preferably, the pressure differential is provided by a pump such as an IV pump. Other exemplary sources of fluid are described in more detail below, and each fluid source may be a means for supplying moisture 72 to the maintaining means 56 of the dressing 50.

The first layer 52 may be variously configured. In exemplary embodiments, the first layer 52 includes a fine mesh absorbent facing with multi-layered cellulose. It has been found that such a configuration promotes maximum absorbency. This material preferably has wicking characteristics, which have been found to be especially desirable in wound dressings to be used on wounds which require draining.

The first layer 52 may for example, be a porous fabric or mesh such as the screen fabrics or filter fabrics of polyethylene, polypropylene, nylon or polyester. Any of a variety of polymeric materials may be used. The choice of suitable material and pore size or the like for transfer in the first layer 52 will be dictated by the needs of the patient. In addition, the material preferably is flexible, soft, conformable, non-irritating, non-sensitizing, and non-cytotoxic.

The first layer 52 may include an antibacterial or other therapeutic agent, and may be configured into a pattern, such as a waffle contour, for greater comfort, and/or with a thicknesses exhibiting desired characteristics of porosity, density, and the like in accord with optimum wound treatment. In particular, a smooth, unwrinkled, compressible layer can significantly contribute toward reduction of bruising. The first layer 52 may include a silver coating (such as that found in the Acticoat® Burn Dressing) that allows continuous application of silver to a wound over a period of time.

Similarly, the wound contacting surface or first layer 52 can be further modified to include therapeutic agents such as biologically active substances to promote healing which are bound to the structure of the wound dressing.

It is intended that the present invention should not be limited by the type, form, or construction of a particular first layer 52.

The middle layer 53 in the illustrated embodiments is a porous material that acts as a filler between the first layer 52 and the third layer 54 to prevent compression forces being applied to the first layer by, for example, the irrigation system 56 or the treating means. The middle layer 53 also may result from a layering or deposition process that forms the various conduits integrally with the middle layer, which will provide flexibility in the cross-section and size of the conduit and gel filled networks to optimize there impact in the dressing. An exemplary cross-section for the conduits in the irrigation system would be conical over the length of a particular conduit in the middle layer with the pores getting closer together and larger along the length of a particular conduit.

The third layer 54 in the illustrated embodiments is a moisture barrier. The primary functions of the third layer 54 are to protect the underlying structures from mechanical damage, from contamination and from a gross loss of liquids.

It may be desirable that the third layer 54 is transparent or does not obstruct a view of the wound. Examples of suitable materials for the third layer 54 are polyolefines, soft acrylic resins, polyurethanes, plasticized PVC, silicone and other synthetic elastomers, and substantially impermeable plastic foil.

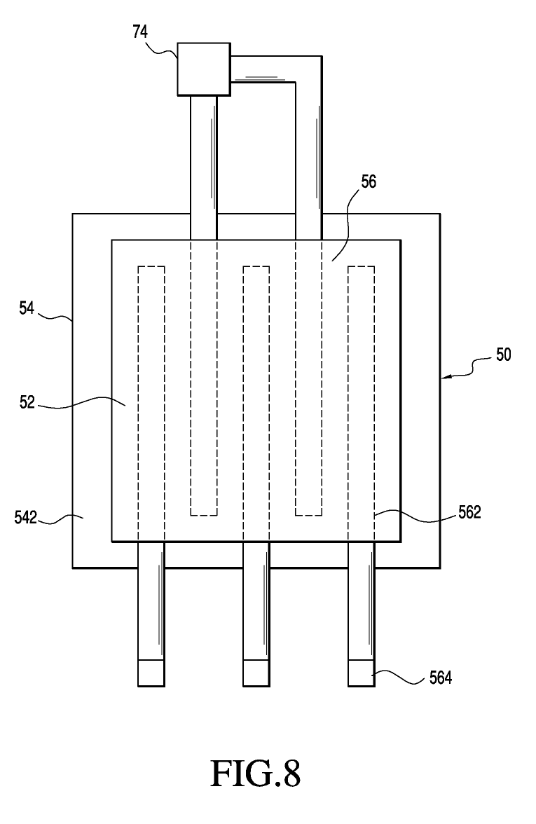

The third layer 54 in at least one exemplary embodiment has a larger footprint area than the other layers in the dressing 50 as illustrated in, for example, FIGS. 3A-3C. As illustrated in, for example, FIG. 8, the extra area can be covered with a suitable skin adhesive 542 for fixing the dressing and sealing the wound. A quantity of adhesive 542 may be included on the exposed peripheral underside of third layer 54. Such adhesives as Monsanto 612 or Flexcon adhesive H-566 are suitable for this purpose. The adhesive 542 should be hypoallergenic and designed for prolonged use on human skin.

The irrigation system 56 preferably includes flexible tubing for the conduit(s) 562. As used herein, the tubing may be any conduit or means which provides fluid communication from a source of fluid to one or more areas in the dressing system 50. The tubing or conduit 562 preferably is a flexible plastic, more preferably plasticized PVC, or other conventional polymer. The construction of such conduits is well known to those skilled in the art.

In accordance with the present invention, the conduit 562 may alternatively be formed of a hydrogel or some other high water content moiety. For example, the conduit 562 may be formed of a hydrophilic polyurethane hydrogel of the composition described in U.S. Pat. No. 5,120,816 (Gould et al.).

There may be a number of tubes 562 providing fluid communication to any individual area within the middle layer 53, and the tubes 562 may be oriented in a number of ways. For example, FIGS. 1A and 6A illustrate a unitary matrix of conduits 562, while FIGS. 2A and 6B illustrate a series of individual conduits 562. In another exemplary embodiment (not shown), the configurations in FIGS. 6A and 6B may be combined where passageways in perpendicular conduits do not communicate with each other. Additionally, the tubes and conduit assemblies may be oriented to define different flow paths such as having fluid provided from more than one side (or portion if the dressing layers are non-polygonal or has non-polygonal areas). Furthermore although one, three, four and five conduits are illustrated in the figures, the number and arrangement of the conduits preferably is selected based upon the size and purpose for the dressing.

The portions of the conduit(s) 562 inside the second layer 53 preferably include one or more holes 5622 so that fluid in the conduit 562 can wet the first layer 54. It is intended that the invention should not be limited by the size or number of holes 5622 in the conduit 562. One skilled in the art will readily recognize that the number and size of the holes 5622 may be dependent on the type of wound, on the size of the dressing, or other factors. It has been found that placing a hole 5622 every centimeter or so provides suitable wetting and fluid dispersion across the first layer 52. The approximate centimeter distance between holes 5622 preferably also is the absolute distance between holes irrespective of a particular conduit 562. FIGS. 9A and 9B, for example, illustrate two exemplary hole patterns for positioning holes 5622 on a particular conduit 562.

The irrigation system 56 preferably includes an access port 564 for delivering and/or removing fluid from the conduit 562 within the first and/or second layers as illustrated, for example, in FIGS. 1A and 1B. The irrigation system 56 may also include multiple access ports 564 as illustrated, for example, in FIGS. 2A and 2B.

The ends of each conduit 562 may be capped or closed, or open or openable. In some embodiments of the invention, the conduit end opposite the access port 564 is sealed and positioned within the second layer 53. In other embodiments of the invention, the conduit end opposite the access port 564 passes through the second layer 53 as illustrated, for example, in FIG. 1C, which illustrates a capped end 565. In this illustrated embodiment, the end 565 preferably is capped and openable, and alternatively may include a fitting such as a Luer-lock for attachment to, for example, other conduits, a container, or a pump. For example, the end 565 of the conduit may be in fluid communication with a vacuum suitable for drawing fluid into the dressing 50, or for drawing fluid or exudate in the dressing 50 out. In an alternative embodiment, a filter may be placed on the outlet conduit to avoid any organisms getting into the system.

A cap, closure, seal, valve, clamp, transfer leg closure, or the like is typically located in or on the conduit. It is intended that the present invention is not limited by the type of material used to construct the conduit. The ends of the conduit may be attached to an infusion set for administering or changing the solution in the conduit as required.

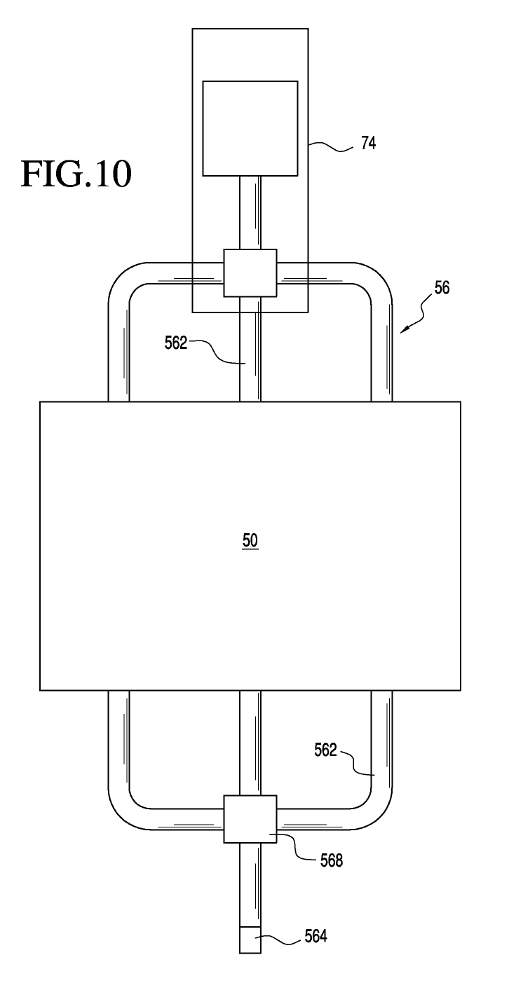

The dressing 50 in another exemplary embodiment is connected to a vacuum system 64 to remove exudates away from the wound. The vacuum system may be connected to the irrigation system 56 (illustrated in FIG. 10) and/or a separate irrigation system 56′ (illustrated in phantom in FIG. 8) for removal purposes. Preferably in this embodiment, the separate conduit system 56′ would mirror the configuration/layout/design of the irrigation system 56.

In another exemplary embodiment, the first layer includes a slow release substance and/or fluid like, for example, an antibiotic and/or silver. Preferably, the added material will be slowly released from the first layer 52. Additionally, the added material may be activated by the moisture present within the first layer 52.

Another exemplary embodiment has the third layer 54 be removable from the rest of the dressing 50 to allow replacement of the third layer 54 to reduce the cost of use. Preferably in this alternative embodiment, the third layer 54 is attached to the rest of the dressing such that it may be easily removable while preventing leakage of fluid from the dressing sides. One possibility is to have the surface area of the second layer attaching to the rest of the dressing with Velcro or some other similar attachment mechanism. In at least one exemplary embodiment, each of the layers may be switched out separate from the other layers.

Another exemplary embodiment illustrated in FIG. 7 includes a feedback system 76 such as an electric resistance feedback system built into at least one layer of the dressing 50. The feedback system 76 preferably monitors the level of moisture in the wound bed. The feedback system 76 may include Drager (or Draeger) tubes built into the dressing 50 to detect certain organisms that may be in the wound bed, to allow the feedback system to instruct a pump 60 to pump antibiotics through the irrigation system 56 into the first layer 52 and/or notify medical personnel of the presence of certain organisms preferably including identification of the type of organism.

In use, the dressing 50 preferably is placed over a wound, and can be held in place by an overlaying elastic garment (not shown), by adhesive tape strips (not shown) bridging from the dressing 50 to surrounding skin, by adhesive double-faced tape pieces, or by any other dressing method as recognized in the art. Alternatively, the dressing 50 may be held in place by adhesive 542 when present. Thus, if the dressing 50 is used to treat the site of a burn, minimal or no adhesive material is used, and the dressing 50 preferably is held in place in any manner that a physician determines as most comfortable for a patient. The dressing 50 can be applied directly to the wound to protect it and to promote moisture retention and wound healing. The dressing 50 can be applied dry or wet, i.e., prior to or after adding fluid to the irrigation system 56.

Once the dressing 50 is in place, a source of fluid such as a bag or syringe containing saline solution may be connected to one or more conduits 562 through at least one port 564. The fluid may then be pumped or drawn into the conduits 562, where the fluid passes through the holes 5622 and wets the first layer 52.

The dressing 50 also may be of sufficient size to cover the entire bed. Alternatively, the dressing 50 may be in the form of a sock and/or mitten to cover an extremity. A further alternative shape for dressing 50 is to be any shape or size to cover any particular body part.

The dressing 50 may also be packaged by sealing into a water-impermeable plastic poach and sterilized by gamma irradiation or other suitable sterilization technique.

As used in this specification and the claims, fluid refers to any gaseous or liquid fluid suitable for contact with a wound. Many of these fluids are well known to those skilled in the art. Exemplary fluids include, but are not limited to air, oxygen, water, distilled water, and a saline solution. Fluid also may include a vapor or liquid solution or suspension containing one or more therapeutically beneficial ingredients, including but not limited to one or more antibiotics, antimicrobials, antipathogenic polypeptides, antibacterial and antifungal agents, antiviral agents, steroidal and non-steroidal anti-inflammatory drugs, anti-cancer drugs, anti-clotting agents, anti-tissue damage agents, immune modulators, monoclonal and polyclonal antibodies, hormones, immunosuppressives, thrombolytic agents, vitamins, amino acids, prostaglandins, enzymes, buffers and salts, anions, preservatives, vasodilators; antiarrhythmic agents, cardiotonics, antihypertensives, local anesthetics, hypotensive diuretics, hypnotics and sedatives, central nervous system agents, antitubercular agents, post-cerebral embolism agents, antiulcer agents, preservatives, humectants such as propylene glycol, antimicrobial agents such as silver, pharmaceutical agents, analgesics, non-stick compositions such as silicore emulsions, wound-healing agents, odor-absorbing agents and fragrances, an aqueous alcohol such as an aqueous ethanol; or a sulfamylon solution.

In use of exemplary embodiments of the invention, the fluid may also include cells or molecules that enhance epidermis growth and/or encourage granulation tissue growth.

In at least some uses of exemplary embodiments of the invention, the fluid is water, more preferably saline solution, for example containing from 0.1 to 5.0, often about 0.9, percent by weight sodium chloride, preferably physiological saline. If desired, the aqueous liquor may contain ions which exhibit physiological activity, for example silver or zinc cations. The saline solution may have hypotonic or hypertonic properties depending on its salt content. In the case of burn patient, the fluid preferably is 5% Sulfamylon® solution (Dow B. Hickman, Inc., Sugarland, Tex. U.S.A.).

The fluid may be any temperature. For example, for some therapeutic applications, it may be desirable to alternate between a hot fluid and a cold fluid. However, in at least one exemplary embodiment the fluid temperature is controlled.

As used in this specification and the claims, adapted for communication, communicating, or similar terms refer to any means, structures, or methods for establishing fluid flow through the system, as are well known by practitioners in the art. For example, a conduit 562 may have a connector 564 adapted to receive or connect to a mated connector on another conduit. As used in this specification and the claims, connector refers to any structure used to form a joint or to join itself to another piece. These connectors or connections establish a fluid flow path through various elements of the apparatus, assembly, or system. Typical connections include but are not limited to mating connections, such as Luer-type, screw-type, friction-type, or connectors that are bonded together.

Although the present invention has been described in terms of particular preferred and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the preferred and alternative embodiments described above can be configured without departing from the scope and spirit of the invention. Therefore, it is to be understood that, within the scope of the appended claims, the invention may be practiced other than as specifically described herein.

### VI. INDUSTRIAL APPLICABILITY

The above-described invention is useful for the treatment of wounds by improving the healing and the time required for healing. The invention is particularly useful in situations where there is insufficient staffing to properly watch and check on the effectiveness of a wound dressing.

We claim:

1. A wound management system comprising:
a dressing having
a first layer,
a second layer abutting said first layer, and
a third layer abutting said second layer,
an irrigation system in communication with said second layer, and
means for treatment in communication with at least one of said second layer and said irrigation system.

2. The wound management system according to claim 1, wherein the treatment means includes
a vibrational energy source,
a conduit system filled with material capable of transferring vibrational waves from the vibrational energy source into at least one of said first layer and said second layer.

3. The wound management system according to claim 2, wherein the treatment means further includes
a light source,
a light distribution system having a plurality of optical fibers embedded in at least one of said irrigation system and said conduit system.

4. The wound management system according to claim 3, further comprising:
a thermistor in said dressing, and
a controller in communication with said thermistor.

5. The wound management system according to claim 4, further comprising:
a heater in communication with said irrigation system and said controller, and
a cooler in communication with said irrigation system and said controller.

6. The wound management system according to claim 4, further comprising a pH electrode system in communication with said controller, said pH electrode system including a plurality of pH electrodes in said dressing.

7. The wound management system according to claim 6, further comprising:
an acidic fluid source in communication with said irrigation system and said controller, and
a base fluid source in communication with said irrigation system and said controller.

8. The wound management system according to claim 1, wherein the treatment means further includes
a light source,
a light distribution system having a plurality of optical fibers embedded in said irrigation system.

9. The wound management system according to claim 1, further comprising:
a thermistor in said dressing, and
a controller in communication with said thermistor.

10. The wound management system according to claim 9, further comprising:
a heater in communication with said irrigation system and said controller, and
a cooler in communication with said irrigation system and said controller.

11. The wound management system according to claim 1, further comprising:
a controller, and
a pH electrode system in communication with said controller, said pH electrode system including a plurality of pH electrodes in said dressing.

12. The wound management system according to claim 11, further comprising:
an acidic fluid source in communication with said irrigation system and said controller, and
a base fluid source in communication with said irrigation system and said controller.

13. A wound management system comprising:
a dressing having
a first layer,
a second layer abutting said first layer, and
a third layer abutting said second layer,
an irrigation system in communication with said second layer,
a light distribution system in communication with said dressing, said light distribution system including a light source providing light having a wavelength between 3 nm and 380 nm, and
a vibrational energy source in communication with said dressing.

14. The wound dressing system according to claim 13, wherein said light distribution system includes a plurality of optical fibers at least partially embedded in said irrigation system.

15. The wound management system according to claim 13, further comprising:
a thermistor in said dressing, and
a controller in communication with said thermistor.

16. The wound management system according to claim 15, further comprising:
a heater in communication with said irrigation system and said controller, and
a cooler in communication with said irrigation system and said controller.

17. The wound management system according to claim 13, further comprising:
a controller,
a pH electrode system in communication with said controller, said pH electrode system including a plurality of pH electrodes in said dressing,
an acidic fluid source in communication with said irrigation system and said controller, and
a base fluid source in communication with said irrigation system and said controller.

18. The wound management system according to claim 13, wherein said vibrational energy source includes a transducer located within said dressing.

19. A wound management system comprising:
treating means for treating the wound,
wound covering means for covering the wound site,
seepage preventing means for preventing seepage from the wound site to the surrounding environment,
feedback means for providing feedback based on conditions within said wound covering means,
wetness maintaining means for maintaining wetness within said wound covering means, and
moisture supplying means for providing moisture to said wetness maintaining means.

20. The wound management system according to claim 19, wherein said treating means includes at least one of
a light distribution system having means for providing light having a wavelength of 3 nm to 380 nm into said wound covering means, and
a vibrational energy source in communication with said wound covering means.
